(19) United States
(12) Patent Application Publication
(10) Pub. No.: US 2015/0132404 A1
(43) Pub. Date: May 14, 2015

(54) Equine feed for the reduction of ulcers in performance horses

(71) Applicant: George Aaron Custer, Ardmore, OK (US)

(72) Inventor: George Aaron Custer, Ardmore, OK (US)

(21) Appl. No.: 14/121,879

(22) Filed: Oct. 30, 2014

Related U.S. Application Data
(60) Provisional application No. 61/962,503, filed on Nov. 8, 2013.

(51) Int. Cl. A61K 31/51; A23K 1/16; A61K 33/00; A61K 33/06; A61K 31/355; A61K 33/30; A61K 33/34; A61K 33/04; A61K 31/07; A23K 1/18; A61K 33/42

(52) U.S. Cl. CPC A61K 31/51; A23K 1/1806; A23K 1/1603; A61K 33/00; A61K 33/06; A61K 33/42; A61K 33/30; A61K 33/34; A61K 33/04; A61K 31/07; A61K 31/355
USPC 424/602; 424/717

(57) ABSTRACT

A feed supplement mixture for performance horses provides a high protein nutritional supply while lowering stress experienced by the horse, reduces the onset of ulcers in the horse's digestive tract and treats ulcers in horses already afflicted with ulcers by the introduction of a quantity of thiamine to reduce stress and sodium bicarbonate to raise the ph level of the feed and the horse's stomach content.

### CROSS REFERENCE TO RELATED APPLICATIONS

Applicant claims the benefit of Provisional Patent Application No. 61/962,503, filed on Nov. 8, 2013, by the same inventor.

### BACKGROUND OF THE INVENTION

1. Field of Invention

A feed supplement mixture for performance horses provides a high protein nutritional supply while lowering stress experienced by the horse, reduces the onset of ulcers in the horse's digestive tract and treats ulcers in horses already afflicted with ulcers by the introduction of a quantity of thiamine to reduce stress and sodium bicarbonate to raising the pH level of the feed and the horse's stomach content.

2. Description of Prior Art

A preliminary review of prior art patents was conducted by the applicant which reveal prior art patents in a similar field or having similar use. However, the prior art inventions do not disclose the same or similar elements as the present horse feed, nor do they present the material components in a manner contemplated or anticipated in the prior art.

In U.S. Patent Application No. 2010/0021430 to Baginski, a feed mixture for treatment of ailments in horses contains a grain product, vegetable oil and vitamin prefix, including some B vitamins, with a reduced amount of sugar and the grain being at least 15% spelt. It allegedly is provided to treat underweight conditions, founder, colic obesity, ulcers, inflammation and chromic disease. In U.S. Patent Application No. 2008/0050455 to Smith, a feed mixture to treat ulcers in horses comprises dry fee with chamomile tea and slippery elm with apples and natural sweeteners. In McCormisk, U.S. Pat. No. 7,601,373, an out balancing feed with multiple vitamin and mineral supplement is provided for improve health and performance.

A medicine, known as a PPI, or proton pump inhibitor, is given to a horse prior to the occurrence of ulcers, as disclosed in U.S. Pat. No. 6,939,881 to Pipers. This medication has been known to be effective on a limited basis and is also known to be extremely costly per dosage. In U.S. Pat. No. 4,960,591 to Gribou, sodium bicarbonate and dry milk is given in tablet form to human beings for stomach distress relief, reacting in the manner of an antacid. A variety of ailments are allegedly treated by a method of feeding a horse carrot juice, carrot pulp in U.S. Pat. No. 6,495,192 to Bezreh. Among those ailments are old age, chewing problems, tooth infections, ulcers, colic, Cushing's, founder, cancer, arthritis, central nervous system disorder, chronic heart disorder and weight loss.

### SUMMARY OF THE INVENTION

Performance horses experience a higher level of stress than other stock horses due to a more active lifestyle. Travel in a stock trailer, boarding in uncommon surroundings and competition events have the same enhanced emotional stress, if not worse, than what humans experience. As a result of this stress, horses are known to develop stomach ailments, especially ulcers, that require long term care, remedial treatments and veterinarian care, which often times takes them out of competition and active participation in events.

Prior art reveals the recognition of this problem and attempts to cure ulcers after onset using drugs known as protein pump inhibitors, primarily Omeprazole. This medication is very expensive and results are not certain. Feeds and feed mixtures are also presented by prior art that attempt to avert the onset of dietary ailments, including underweight conditions, founder, colic, obesity, ulcers, inflammation and chronic disease by the introduction of spelt, vegetable oils and vitamin premix, which contain active probiotics, vitamins, digestive enzymes and amino acids, including thiamine.

The primary objective of the invention is to provide a stable horse feed with mild additives for the treatment of ulcers in performance horse. A secondary objective is to provide the feed for the treatment of performance horses who already have an onset of ulcers. A third objective is to provide the ulcer treatment in an expensive medical format, instead using naturally occurring additives which serve to treat the ulcer without questionable side effects.

### DESCRIPTION OF THE DRAWINGS

There are no drawings.

### DESCRIPTION OF THE PREFERRED EMBODIMENT

A dry horse feed supplement for the prevention and treatment of ulcers in horses, especially those involved in performance activities, provides a feed supplement mixture comprising a quantity of forage or filler product; a quantity of grain product, a quantity of protein product, a quantity of roughage product, essential vitamins and trace minerals, a yeast product, fat, molasses, a measured quantity of thiamine (Vitamin B1) and a measured quantity of sodium bicarbonate. The feed supplement mixture is fed to a horse based upon the total weight of the horse, in an amount between 0.50-1.50% of the body weight, depending upon the level of work and activity stress of the horse.

The introduction of the combination of thiamine and the sodium bicarbonate causes no harm to the horse and is ingested without observed noticeable difference in consumption by a horse of other feed mixture supplements. It has been observed in testing of the product that the thiamine has a calming effect on horses, which in turn decreases the stress levels. It has also been observed that the introduction of the sodium bicarbonate at the recommended levels acts as a buffer and maintains an optimal ph level in the digestive tract, much as experience with the consumption of bicarbonates by humans in antacids and stomach treatment medications. The combination of the two in a singular feed mixture supplement promotes a more calm and trainable horse as well as a horse that reacts better to the stress associated with performance and competition.

A preferred mixture of the ingredients comprising the stress reducing feed supplement mixture is presented in the range a follows:

| Ingredient | Quantity by Percentage |
|---|---|
| forage products | 35-40% |
| grain products | 12-25% |
| protein products | 10-30% |
| roughage products | 25-50% |
| vitamins and trace minerals | ≦0.5% |
| yeast | ≦0.3% |
| fat | 3-10% |
| molasses | 2-5% |
| sodium bicarbonate | 0.2-1.0% |
| thiamine (B1) | 0.2-1.0% |

The ingredient total cannot exceed 100.0% and the mixture should be suited by final composition based upon the relative level of activity, weight composition and other dietary considerations rendered by the physical environment, common grazing materials, other feed supplements consumed by the horse, available water supply and other subjective aspects of the individual horse or the competition herd of horses.

Unlike any prior art feeds, which only include thiamine by passing reference in a list of vitamins and not requiring thiamine as an ingredient at all (McCormick, Column 6, lines 63-67, wherein thiamine is considered a non-essential component of the feed supplement and can be omitted from the feed product), thiamine is an essential element in the present feed supplement mixture and actually is provided to produce an effect on the horse beyond general diet. Performance improvement using the present horse feed is based upon the treatment of the horse's dietary concerns and the lowering of over-all stress experienced by the horse. Sodium bicarbonate has not been observed in the prior art as it pertains to use in horses or performance animals, although sodium bicarbonate is a known antacid or stomach acid treatment ingredient in humans. See, Gribou, supra.

For those horses already experiencing ulcers, the feed supplement mixture is an appropriate treatment supplement and is greatly less costly than treatment of a veterinarian using known methods of treatment, Omeprazole which is a protein pump inhibitor (Smith and Pipers). Treatment using this product can cost as much as $800.00 per month, depending upon dosage administered, plus the cost of the veterinarian. In contrast, the stress reducing and ulcer preventing feed supplement mixture is approximately the same cost as most other feed supplement mixtures, as the addition of the thiamine and the sodium bicarbonate add but a nominal amount to the cost of production of the feed supplement, sold in bulk or in bags.

It would be better to deliver the present horse feed supplement containing the sodium bicarbonate and thiamine in measured quantity weather sealed bags. Water has been known to break down sodium bicarbonate, and such water introduction should be limited to the time of consumption of the food product. It is recommended also that when consumption of the feed supplement mixture by the horse occurs that plenty of water is available to optimize the anti-acidic reactions which occur within the stomach of the horse. As to the thiamine component, it is recommended that avoidance of sulfites within the mixture is followed, since sulfites, which are generally found in food additives, have been known to affect absorption of thiamine during digestion. Sulfites and flavonoids have been found to oxidize the thiazole rings within thiamine, rendering the thiamine unabsorbable during digestion.

The feed composition ingredients, delivered in the parameters set above with regard to content quantity by percentage includes the ingredients list, in order of most ingredient quantity to least ingredients quantity, provides the feed containing no less than 14% crude protein, 6.5% crude fat, a no more than 22% crude fiber, no less than 0.6% and no more than 1% calcium, no less than 0.3% phosphorus, as well as quantities of zinc, copper, selenium, Vitamin A and Vitamin E. There are special blends suited for older performance horses and other blends for various younger performance horses. It is also contemplated that a lower fat content may be considered in the mixture, but less than the range indicated is not considered optimal due to the activity of the horse making a lower fat content less than desirable. The feed is fed at a rate of 0.5% to 1.5% of the animal's body weight as a supplement to good quality grass hay and/or pasture fee choice, with the animal having access to an abundant supply of fresh, clean water.

Not only will an improvement be realized in a more calm demeanor of the horse, reduced stress, and increased appetite. Improved health is also seen in the animals main, tail, and coat as well as improved strength and durability of the animal's hoof, evident of the enhanced protein in the animal's diet. In those horses observed having been fed the present feed supplement, better statistical performance results have been obtained in the form of increase speed, stamina and overall musculature and fitness, especially in competition horses with the combined presence of the thiamine, to reduce the stress level of the horse, and sodium bicarbonate to reduce the stomach acid of the horse by raising the pH of the feed mixture caused by stress and higher acidic content of other feeds at the food source, not contemplated by the prior art, alone or in combination.

While the invention has been particularly shown and described with reference to a preferred embodiment thereof, it will be understood by those skilled in the art that changes in form and detail may be made therein without departing from the spirit and scope of the invention.

What is claimed is:

1. A dry horse feed supplement for the prevention and treatment of ulcers in horses and reduction of stress, especially those involved in performance activities, comprising:
a quantity of forage or filler product;
a quantity of grain product;
a quantity of protein product;
a quantity of roughage product;
essential vitamins and trace minerals;
a yeast product;
fat;
molasses;
a measured quantity of thiamine (Vitamin B1) for the reduction of stress in the horse; and
a measured quantity of sodium bicarbonate to raise the pH of the feed for the prevention, reduction and treatment of ulcers caused by feed content and stress in the horse.

2. The horse feed supplement, as disclosed in claim 1, wherein the ingredients within the supplement further comprising:
35-40% forage products;
12-25% grain products;
10-30% protein products;
25-50% roughage products;
≦0.5% vitamins and trace minerals;
≦0.3% yeast;
3-10% fat;
2-5% molasses;
0.2-1.0% sodium bicarbonate; and
0.2-1.0% thiamine (B1), wherein the ingredient total cannot exceed 100.0%, with the mixture of ingredients suited by final composition based upon the relative level of activity, age of the animal, weight composition and other dietary considerations rendered by the physical environment, common grazing materials, other feed supplements consumed by the horse, available water supply and other subjective aspects of the individual horse or the competition herd of horses.

3. The horse feed supplement, as disclosed in claim 1, wherein the feed composition ingredients, delivered in the parameters set above with regard to content quantity by percentage comprising:
no less than 14% crude protein, no less than 6.5% crude fat, a no more than 22% crude fiber, no less than 0.6% and no more than 1% calcium, no less than 0.3% phosphorus, and quantities of zinc, copper, selenium, Vitamin A and Vitamin E, having various blends within the parameters above specifically and particularly suited for older performance horses, younger performance horses and competition performance horses.
